(19) United States
(12) Patent Application Publication
(10) Pub. No.: US 2004/0265917 A1
(43) Pub. Date: Dec. 30, 2004

(54) Methods of measuring the ability of a test compound to inactivate a biological target in cells of a subject

(76) Inventors: Dennis Benjamin, Acton, MA (US); Charles Thompson, Stow, MA (US); Bryan Wang, Wayne, PA (US); James Wakefield, Arlington, MA (US); Malcolm L. Gefter, Lincoln, MA (US); Christopher C. Arico-Muendel, West Roxbury, MA (US)

Correspondence Address: LAHIVE & COCKFIELD, LLP., 28 STATE STREET, BOSTON, MA, 02109, US

(21) Appl. No.: 10/820,530

(22) Filed: Apr. 7, 2004

Related U.S. Application Data
(60) Provisional application No. 60/460,920, filed on Apr. 7, 2003.

(51) Int. Cl. G01N 33/53; G01N 33/567

(52) U.S. Cl. 435/7.2

(57) ABSTRACT

The present invention provides a method of assessing the ability of a compound (the “test compound”) which is an inhibitor of a biological target to inhibit the biological target in a biological compartment of interest when administered to a subject in vivo.

### RELATED APPLICATIONS

This application claims priority to U.S. Provisional Patent Application Ser. No. 60/460,920, filed on Apr. 7, 2003, the entire contents of which are incorporated herein by reference.

### BACKGROUND OF THE INVENTION

The process of drug discovery often involves the identification of compounds which bind to and modulate the activity of a biological target molecule. For example compounds which are identified by initial screens as ligands for the target can then be assessed for their ability to modulate the activity of the target in an in vitro cell-based or cell-free assay. But while determining the in vitro activity of drug candidates is in most cases straightforward, the ability of a drug candidate to affect the target in the biological compartment of interest when administered to a subject in vivo is much more difficult to determine. However, such information can be particularly valuable for determining the appropriate dose and dosing schedule of a drug candidate and for correlating the effect on the biological target with observed clinical effect.

One biological target of current interest is methionine aminopeptidase-2, an enzyme that catalyzes the post-translational cleavage of the N-terminal methionine residue from a variety of proteins. The enzyme is the molecular target of the fungal metabolite fumagillin, which, along with a variety of analogs, has been shown to halt the growth and division of endothelial cells and to have anti-angiogenic activity. Methionine aminopeptidase-2 is, therefore, of interest as a molecular target for the discovery of compounds which can be used to treat diseases associated with aberrant angiogenesis, such as solid tumors.

The development of compounds which inhibit methionine aminopeptidase-2 and other biological targets as therapeutic agents would be assisted by methods which allow the measurement of the in vivo effect of such compounds on the molecular target. Thus, there is a need for a method of determining the effect on the activity of a biological target, such as methionine aminopeptidase-2, in a target tissue, of an inhibitor of the biological target administered in vivo.

### SUMMARY OF THE INVENTION

The present invention provides a method of assessing the ability of a compound (the “test compound”) which is an inhibitor of a biological target to inhibit the biological target in a biological compartment of interest when administered to a subject in vivo. In particular, the method enables the determination of the amount or fraction of the biological target in a biological sample which has not been inactivated by the test compound. The method comprises the steps of (1) administering the test compound to a subject, such that any of the biological target in the subject's body which reacts with the test compound is inactivated and any of the biological target which does not react with the test compound is free; (2) removing a biological sample comprising one or more cell types from the subject; (3) determining the amount of free biological target within the biological sample or a fraction thereof; and, optionally, (4) comparing the amount determined in step (3) with the amount of free biological target in a control sample. A decrease in the amount of free biological target determined in step (3) compared to the amount determined in the control sample provides a measure of the amount of inactivated biological target in the biological sample or fraction thereof.

In one embodiment, the biological target is methionine aminopeptidase-2 (hereinafter also referred to as “MetAP-2”), and the invention provides a method of assessing the ability of a test compound which is an inhibitor of MetAP-2 to inhibit MetAP-2 activity in a biological compartment of interest when administered to a subject in vivo. In particular, the method enables the determination of the amount or fraction of MetAP-2 in a biological sample which has not been inactivated by the test compound. The method comprises the steps of (1) administering the test compound to a subject, such that any MetAP-2 in the subject's body which reacts with the test compound is inactivated MetAP-2 and any MetAP-2 which does not react with the test compound is free MetAP-2; (2) removing a biological sample comprising one or more cell types from the subject; (3) determining the amount of free MetAP-2 in the biological sample or fraction thereof, and, optionally, (4) comparing the amount determined in step (3) with the amount of free MetAP-2 in a control sample. A decrease in the amount of free MetAP-2 determined in step (3) compared to the amount determined in the control sample provides a measure of the amount of inactivated MetAP-2 in the biological sample or fraction thereof.

In one embodiment, the amount of free biological target, such as MetAP-2, in the biological sample or fraction thereof is determined by a method comprising the steps of (i) contacting the biological sample or a fraction thereof with a saturating amount of a quantifiable irreversible inhibitor of the biological target, so that substantially all of the free biological target reacts with the quantifiable irreversible biological target inhibitor to form a target/inhibitor complex; and (ii) determining the amount of target/inhibitor complex formed in step (i).

The test compound can be any compound which is, or is thought likely to be, an inhibitor of the biological target. Preferably, the test compound has been shown to be an inhibitor of the biological target in a in vitro assay, such as a cell-free or cell-based assay. The test compound is preferably a compound which is an active site-directed inhibitor of the biological target or a compound which binds to the biologically relevant ligand binding site of the biological molecule. The test compound can also be a compound which inhibits the biological target, for example, via an allosteric effect, by binding to the biological target at a site other than the active site.

In another embodiment, the invention provides a method for determining the amount of an irreversible inhibitor of a biological target, such as MetAP-2, in a biological sample. The method comprises the steps of (1) contacting the biological sample with a saturating amount of the biological target, such that substantially all of the irreversible inhibitor of the biological target reacts with the biological target to inactivate the biological target, while any biological target which does not react with the irreversible inhibitor is free biological target; (2) determining the amount of free biological target; and (3) comparing the amount of free biological target with the amount of biological target added in step (1), whereby a decrease in the amount measured in step (2) compared to the amount measured in step (1) provides a measure of the amount of the irreversible inhibitor in the biological sample.

In another embodiment, step (3) above is substituted by the step of comparing the amount of free biological target to the amount of free biological target in a control biological sample. The control biological sample is a sample identical to the biological sample, but is derived from a subject or an in vitro system to which the irreversible inhibitor has not been administered. The control biological sample also has been contacted with biological target in a manner substantially identical to step (1) of the above method.

In one embodiment, the amount of free biological target is determined by measuring the activity of the biological target in the biological sample. In another embodiment, the amount of free biological target is determined by a method comprising the steps of (i) contacting the biological sample with a saturating amount of an irreversible quantifiable inhibitor of the biological target, such that substantially all of the free biological target reacts with the irreversible quantifiable inhibitor to form a target/inhibitor complex; and (ii) determining the amount of target/inhibitor complex produced in step (i). A decrease in the amount of complex formed compared to the amount of enzyme added to the sample in step (i) is a measure of the amount of inactivated biological target and, hence, of the amount of the irreversible inhibitor in the biological sample.

### BRIEF DESCRIPTION OF THE DRAWINGS

FIG. 1 illustrates the quantification of the MetAP-2-inhibitor complex in one embodiment of the invention.

FIG. 2 is a graph illustrating the free MetAP-2 Levels in white blood cells of female Sprague-Dawley rats after a single dose of Compound 2.

FIG. 3 is a graph showing the free MetAP-2 levels in white blood cells, liver, spleen, lymph nodes and thymus of male and female Sprague-Dawley rats after a single dose of Compound 2.

FIG. 4 illustrates free MetAP-2 levels in tissues relative to those in white blood cells of male and female Sprague-Dawley rats after a single dose of Compound 2.

FIG. 5 presents graphs illustrating results of an ELISA-based assay and a gel shift assay, both of which show a dose-dependent decrease in free MetAP-2 levels in tumor and liver tissue from mice bearing murine melanoma tumors treated with vehicle PO, 3 mg/kg Compound 2 every other day PO or 30 mg/kg Compound 2 every other day PO.

### DETAILED DESCRIPTION OF THE INVENTION

The present invention provides methods for determining the effect of a test compound, administered to a subject in vivo, on the activity level of a biological target in a particular tissue or cell population or other biological compartment of the subject. Specifically, the method allows the determination of the extent of inactivation of the biological target within a particular biological compartment or cell type by the test compound. The method can be used, for example, to assess the ability of the test compound to inhibit the activity of the biological target within a tissue or cell type of interest. This information can be used to identify compounds which are effective inhibitors of the biological target in vivo. The method can also be used to assess the response of a subject, such as a patient suffering from a condition treatable with an inhibitor of the biological target, to a particular test compound, for example, a test compound which is a drug or drug candidate. The method can also be used to evaluate different routes of administration of the test compound in vivo and/or to optimize the dosing amount and frequency of the test compound.

In a first embodiment, the method of the invention comprises the steps of: (1) administering a test compound to the subject, such that the biological target in the body of the subject which reacts with the test compound is inactivated biological target and any biological target that does not react with the test compound is free biological target; (2) removing a biological sample comprising one or more types of cells from the subject; (3) determining the amount of free biological target in the sample or a fraction thereof; and, optionally, (4) comparing the amount of free biological target determined in step (3) with the amount of free biological target in a control sample.

The biological target can be any biological molecule which is a target, or potential target, of pharmacotherapy. For example, the biological target can be a biological molecule which has been implicated in the initiation or progression of a disease. The biological target can be, for example, a peptide, a protein or a nucleic acid. Preferably, the biological target is a protein. For example, the biological target can be a cytokine; a receptor, such as a G-protein-coupled receptor, including CCR5, CXCR4, the somatostatin receptors, and the GnRH receptor; a nuclear transcription factor, such as the androgen receptor, the estrogen receptor, NFkB or NFAT; a receptor kinase, such as EGFR, VEGFR, insulin-like growth factor receptor and Her-2/Neu; a polyDNA molecule, or an RNA molecule. Other suitable biological targets include enzymes, such as a kinase, for example a tyrosine or serine/threonine kinase; thymidylate synthase; cyclooxygenase, e.g. prostaglandin G synthase, prostaglandin H synthase; a protease, such as a serine proteases, for example, trypsin; and penicillin binding proteins. In a preferred embodiment, the biological target is MetAP-2.

For the purposes of the present invention, a compound “reacts with” a biological target when it binds to the target. The compound can bind to the target via formation of a covalent bond between the compound and the target, or it can bind non-covalently, for example, via ionic interactions, hydrophobic interactions, polar interactions, hydrogen bonding, or a combination of two or more of these types of interactions.

In one embodiment, the amount of free biological target is measured using a assay, such as an activity assay or a binding assay. For example, the ability of a receptor to bind its endogenous ligand can be used to determine the amount of free receptor in the sample. When the biological target is an enzyme, for example, the enzymatic activity of the sample can also be determined using standard activity assays.

In another embodiment, the step of determining the amount of free biological target in the biological sample (step (3)) is accomplished by a method comprising the steps of (i) contacting the biological sample or a fraction thereof with a saturating amount of a quantifiable irreversible inhibitor of the biological target, whereby substantially all of the free biological target in the biological sample reacts with the quantifiable irreversible inhibitor to form a target/inhibitor complex; (ii) determining the amount of target/inhibitor complex produced in step (i). In this embodiment, the step of comparing the amount of free biological target determined in step (3) with the amount of free biological target in a control sample (step (4)) is accomplished by a method comprising the step of comparing the amount of target/inhibitor complex determined in step (ii) with the amount of target/inhibitor complex formed in a control biological sample, wherein a decrease in the amount of target/inhibitor complex determined in step (ii) compared to the amount formed in the control biological sample provides a measure of the extent of inactivated biological target in the biological sample.

In a preferred embodiment, the invention provides a method for determining the ability of a test compound to inactivate MetAP-2 in one or more cell types in a subject when administered to the subject in vivo. The method comprises the steps of (1) administering the test compound to the subject, such that MetAP-2 in the body of the subject which reacts with the test compound is inactivated MetAP-2 and any MetAP-2 that does not react with the test compound is free MetAP-2; (2) removing a biological sample comprising one or more types of cells from the subject; (3) determining the amount of free MetAP-2 in the biological sample or fraction thereof; and, optionally (4) comparing the amount of free MetAP-2 determined in step (3) with the amount of free MetAP-2 in a control sample.

In one embodiment, the amount of free MetAP-2 in the biological sample or fraction thereof is determined by measuring the MetAP-2 enzyme activity in the sample. Given that enzyme activity correlates with the amount of active enzyme present, the amount of free enzyme may be determined in this way. Methods for measuring MetAP-2 activity are known in the art and include, for example, the method taught in U.S. Pat. No. 6,261,794, incorporated herein by reference in its entirety.

In another embodiment, the amount of free MetAP-2 in the biological sample or fraction thereof is determined by a method comprising the steps of (i) contacting the biological sample or fraction thereof with a saturating amount of a quantifiable irreversible MetAP-2 inhibitor, whereby substantially all of the free MetAP-2 in the biological sample reacts with the quantifiable irreversible Metap-2 inhibitor to form a MetAP-2/inhibitor complex; and (ii) determining the amount of MetAP-2-inhibitor complex produced in step (i). The amount determined in step (ii) can be compared to the amount of MetAP-2/inhibitor complex formed in a control biological sample, wherein a decrease in the amount of MetAP-2/inhibitor complex determined in step (ii) compared to the amount formed in the control biological sample provides a measure of the extent of inactivated MetAP-2 in the biological sample.

In the present method, the test compound can be administered to the subject by any suitable route. If the test compound inactivates a fraction of the MetAP-2 molecules within a biological compartment of interest, that biological compartment will include inactivated MetAP-2 molecules and, if the test compound does not inactivate every MetAP-2 molecule in the compartment, the biological compartment will also include free MetAP-2. “Inactivated MetAP-2”, as this term is used herein, refers to MetAP-2 molecules which have reacted with the test compound and are, therefore, unable to react with the quantifiable MetAP-2 inhibitor. “Free MetAP-2”, as this term is used herein, refers to MetAP-2 molecules that have not been deactivated by reaction with the test compound and are, therefore, able to react with the quantifiable MetAP-2 inhibitor. Reaction of free MetAP-2 with the irreversible quantifiable MetAP-2 inhibitor produces a MetAP-2/inhibitor complex. The amount of MetAP-2/inhibitor complex formed is then determined and, optionally, compared to the amount of complex formed in a control sample. A decrease in the amount of complex formed following administration of the test compound compared to the control is ascribed to the presence in the test sample of inactivated MetAP-2 and thereby provides a measure of the extent of inactivation of MetAP-2.

The amount of such complex formed can be compared in one embodiment to the amount of such complex formed in a control biological sample, for example, a sample removed from the subject prior to administration of the test compound but otherwise identical to the biological sample of interest; or total MetAP-2 protein can be quantified and the fraction of complex formed relative to the total MetAP-2 in the sample can be determined. The method thus, allows the determination of the fraction of total MetAP-2 with a particular tissue or cell type of the subject is inactivated by the test compound. At one extreme, in vivo administration of the test compound inactivates substantially all of the MetAP-2 in the cells or tissue from which the biological sample is derived. In this case the amount of complex formed will be small compared to the total MetAP-2 protein in the biological sample. At the other extreme, the test compound inactivates little to no MetAP-2 in the cells or tissue from which the biological sample is derived. In this situation, the amount of complex formed will approach the total MetAP-2 protein within the sample.

For example, prior to administering the test compound to the subject, a control biological sample is removed from the subject. The control biological sample is identical to the biological sample removed following administration of the test compound and is processed or fractionated in a substantially identical manner. The control biological sample, or an appropriate fraction thereof, is contacted with the quantifiable irreversible MetAP-2 inhibitor. The amount of MetAP-2-irreversible inhibitor complex thus formed in the control sample is then measured and compared to result determined in step (4). A decrease in the amount of complex measured for the biological sample or fraction thereof following administration of the test compound compared to the amount measured for the control biological sample or fraction thereof is then ascribed to inactivation of some portion of total MetAP-2 within the biological sample by in vivo administration of the test compound.

In one embodiment, the result determined in step (3) is compared to the result obtained from one or more otherwise identical biological samples obtained from one or more control animals that have not been exposed to the test compound. Prior to removal of the biological sample, a placebo or vehicle control can be administered to the control animal or animals, preferably via the same route of administration used for the test compound. The biological sample is preferably removed from the control animal or animals and processed in a manner which is identical to the removal and processing of the biological sample from the test animal.

In another embodiment, the total MetAP-2 in the biological sample is determined and compared to the amount of complex formed. The total amount of MetAP-2 protein in the sample can be determined, for example, using an antibody specific for MetAP-2 and a method of determining the amount of the complex between this antibody and the protein, such as an enzyme-linked immunosorbent assay (ELISA). It is generally assumed herein that the total MetAP-2 protein in a sample is the sum of the inactivated MetAP-2 and the MetAP-2/inhibitor complex. Thus, a comparison of the amount of complex formed compared to the total amount of MetAP-2 protein provides a measure of the amount of MetAP-2 which was inactivated by the test compound.

In yet another embodiment, the control biological sample is removed from the test subject prior to administration of the test compound to the subject. In this embodiment, the control biological sample is preferably removed from the subject and processed in a manner which is identical to the removal and processing of the test biological sample from the subject. Both the control and test biological samples are then subjected to a saturating amount of the quantifiable inhibitor, and the amount of complex formed is compared in the two cases. A decrease in the amount of complex formed in the test sample compared to the amount formed in the control sample provides a measure of the inactivation of MetAP-2 in the test sample by the test compound.

The test compound can be any compound for which the assessment of in vivo inhibitory activity is desired. Preferably, the test compound has the ability to inhibit the biological target in vitro.

In vitro MetAP-2 inhibitory activity can be determined using methods known in the art, such as, for example, the assay disclosed in U.S. Pat. No. 6,261,794. A variety of compounds which inhibit MetAP-2 activity are known. Suitable MetAP-2 inhibitors include the fumagillin derivatives set forth in U.S. Pat. Nos. 6,207,704; 6,063,812; 6,040,337; 5,204,345; 5,789,405; 5,180,735; 5,180,738; 5,166,172; 5,164,410; and published PCT applications WO 99/61432; WO 02/05804; WO 02/42295; WO 99/59987; and WO 99/59986.

Preferably the test compound binds tightly to the biological target. More preferable, the test compound is an irreversible inhibitor of the biological target. An “irreversible inhibitor”, as this term is used herein, is a compound which inhibits the biological target and has a rate of dissociation from the biological target which is slow relative to the length of time required to complete the assay. For example, if the test compound dissociates from the biological target at a rate k, then 50% of the originally inactivated biological target will remain inactivated at about time 0.69302/k. It is thus preferred that the assay be completed in a time period, t, of less than about 0.7/k, 0.6/k, 0.5/k, 0.4/k, 0.3/k, 0.2/k or 0.1/k. In one embodiment, the irreversible inhibitor reacts with the biological target to form a covalent bond.

When the biological target is MetAP-2, the test compound preferably interacts with the active site of the MetAP-2 enzyme, such that, once a molecule of the test compound contacts a molecule of MetAP-2, it resides in the active site of the enzyme and blocks the reaction of the MetAP-2 molecule with another inhibitor molecule. The test compound can also be a compound which inhibits MetAP-2 by binding to a site on MetAP-2 other than the active site. Preferably, the test compound is an irreversible inhibitor of MetAP-2. Such a compound inhibits MetAP-2 enzymatic activity and dissociates from the enzyme sufficiently slowly such that on the time scale of the method of the invention, very little of it would be expected to dissociate from the enzyme. Suitable irreversible inhibitors of Metap2 include covalent inhibitors of MetAP-2.

A “covalent inhibitor of MetAP-2” is an irreversible inhibitor which reacts with a functional group in the active site of the MetAP-2 molecule to form a covalent bond linking the inhibitor to the enzyme. Suitable examples of covalent inhibitors of MetAP-2 include ovalicin, fumagillin, fumagillol and fumagillin analogues, as described above.

A “saturating amount” as this term is used herein, refers to an amount of a compound which is in excess, on a per mole basis, relative to a specified reaction partner. For example, an irreversible quantifiable MetAP-2 inhibitor is present in a saturating amount if it is present in molar excess over the anticipated amount of free MetAP-2. The irreversible quantifiable MetAP-2 inhibitor can, for example, be present at a 1.1- to 10-fold molar excess over the anticipated amount of free MetAP-2. The anticipated amount of free MetAP-2 can, for example, be determined using the amount of MetAP-2/inhibitor complex formed in a control sample. Alternatively, the irreversible quantifiable MetAP-2 inhibitor can be titrated, with the amount of MetAP-2/inhibitor complex determined as more inhibitor is added. A saturating amount of the irreversible quantifiable MetAP-2 inhibitor is present when the addition of more irreversible quantifiable MetAP-2 inhibitor no longer results in an increase in the amount of MetAP-2/inhibitor complex formed. In a preferred embodiment, in the presence of a saturating amount of the irreversible quantifiable inhibitor, substantially all the free biological target in the sample is converted to target/inhibitor complex. For the operation of the inventive method, it is not necessary that every molecule of free biological target is converted to target/inhibitor complex, but the amount converted to the complex should be greater than the amount which remains free, i.e., more than about 50% of the free biological target should be converted to target/inhibitor complex, preferably at least about 60%, more preferably at least about 75% and most preferably, at least about 90%.

The test compound can be administered to the subject via any suitable route, such as parenteral, including intramuscular, intravenous, subcutaneous and intraperitoneal injection; or the buccal, oral, vaginal, rectal, ocular, intraocular, intranasal, topical, intradermal or transdermal route. The test compound can be formulated for administration using methods known in the art and preferably in a manner which is consistent with the chemical properties of the test compound and the intended route of administration.

A “biological compartment”, as this term is used herein, is a portion of a subject's body and can be, for example, an organ or collection of organs, a tissue or collection of tissues, or a cell or collection of cells or cell types. The biological sample can include any organ, tissue, cells or combination thereof removed from the subject and, in one embodiment, is a tissue or cell type(s) in which the test compound is expected to exert at least part of its therapeutic effect. For example, the biological sample or fraction thereof can be whole blood, a blood fraction or a particular collection of blood cells, such as erythrocytes, white blood cells, T-cells, B-cells, macrophages, or other professional antigen-presenting cells; leukemic cells, lymphoma cells, tumor tissue; cancer cells; bone marrow; synovium, synovial fluid, cerebrospinal fluid, skin, liver tissue or cells, heart tissue, lung tissue, brain tissue, muscle tissue, bone, epithelium, endothelium, prostate tissue, breast tissue, lymph nodes, and spleen. In one embodiment, the biological sample is processed prior to contacting it with the quantifiable inhibitor. Such processing includes methods known in the art and can include, for example, isolation of a particular cell type from within the biological sample, tissue homogenization, and cell lysis. Preferred biological samples or fractions thereof include white blood cells, liver, lymph nodes and spleen.

A “quantifiable inhibitor”, as this term is used herein, is a molecule comprising a (1) a moiety which interacts with the biological target to inhibit the biological target (“binding moiety”) and (2) a moiety that allows the immobilization or quantitation of the inhibitor or an inhibitor/biological target complex (“quantification moiety”). Preferably, the binding moiety binds to the biological target at the same site as the test compound. In this embodiment, reaction between a molecule of the biological target and the test compound prevents a subsequent reaction between the molecule of the biological target and the quantifiable inhibitor. Suitable quantification moieties include a biotin moiety; a methotrexate moiety: a radioisotope, such as tritium or ¹²⁵I; a fluorescent moiety, such as fluoroscein; an antibody, for example, covalently attached to the moiety which interacts with the biological target; single-stranded oligonucleotides, and others as are known in the art. Examples of targets and suitable binding moieties include thymidylate synthase/dideazafolate derivatives; cyclooxygenase/acetylsalicylic acid; serine proteases/phenylmethylsulfonyl fluoride and N-α-p-tosyl-L-lysine chloromethyl ketone; penicillin binding proteins/penicillin.

In one embodiment, the target/inhibitor complex is separated from any unreacted quantifiable inhibitor using a suitable technique, for example, a technique that separates molecules on the basis of size, such as size exclusion chromatography and gel electrophoresis. For example, when the quantification moiety is a fluorescent moiety, the fluorescence intensity of the resulting target/inhibitor fraction can be used to determine the amount of complex present. Similarly, if the quantification moiety is a radioisotope, the level of radioactivity of the target/inhibitor fraction can be used to quantitate the amount of complex formed.

A “quantifiable MetAP-2 inhibitor”, is an irreversible quantifiable inhibitor of MetAP-2, as described above. Preferred quantifiable MetAP-2 inhibitors are covalent MetAP-2 inhibitors. Particularly preferred quantifiable MetAP-2 inhibitors are fumagillin analogues which include a quantification moiety.

The subject can be any animal in which information on the effect of the test compound is desired. Preferably, the subject is a mammal, such as a rodent, dog, cat, horse, cow, sheep or pig, or a primate, such as a non-human primate, such as a monkey or an ape, or a human. In one embodiment, the subject is a laboratory animal, preferably a mouse or a rat. The subject can also be a laboratory animal which has been manipulated, genetically or otherwise, to develop symptoms similar to those of a human disease, such as cancer, including solid tumors and blood cancers, rheumatoid arthritis or other diseases associated with uncontrolled or otherwise undesirable angiogenesis and/or inflammation.

In one embodiment, the quantifiable MetAP-2 inhibitor is a fumagillin analogue of the general structure I, below,

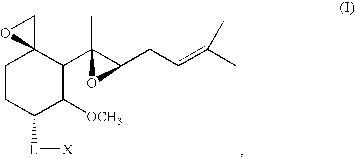

wherein L is a linker group and X is a biotinyl moiety. L can be any moiety which is suitable for linking the biotin moiety to the fumagillin core. Examples of suitable quantifiable Metap2 inhibitors include the biotin-fumagillin conjugate disclosed by Griffith et al. (Proc. Natl. Acad. Sci. USA 95: 15183-15188 (1998); Chem. Biol. 4: 461-471 (1997)) and Sin et al., (Proc. Natl. Acad. Sci. USA 94: 6099-6103 (1997)), each of which is incorporated herein by reference in its entirety. A preferred compound of formula I is the compound of formula II:

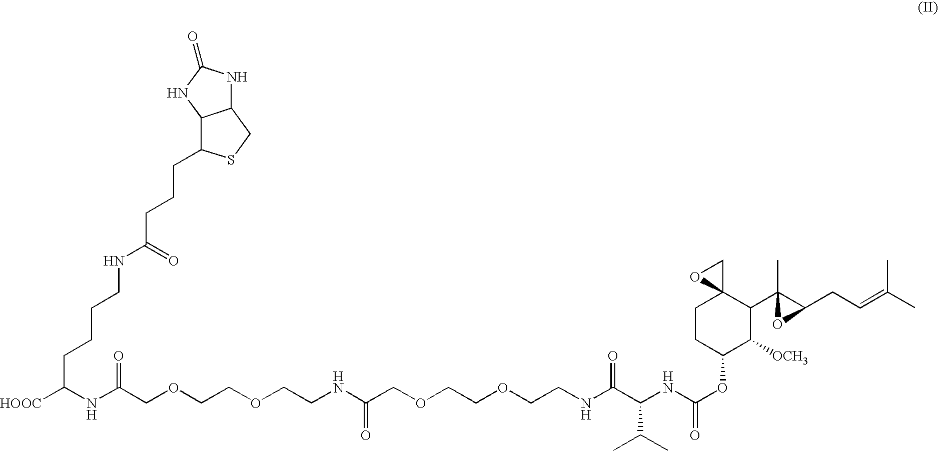

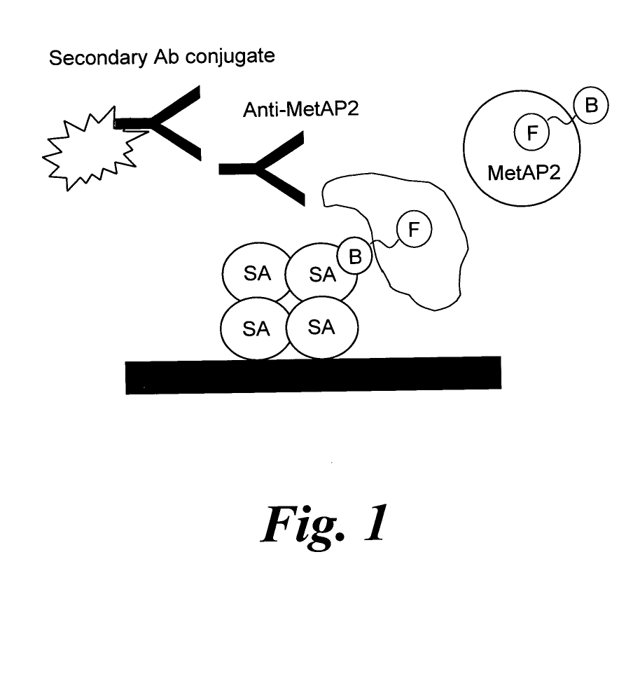

The amount of MetAP-2/inhibitor complex formed can be determined using a variety of methods, such as, for example, the protocol set forth in Example 2. In one embodiment, the complex is immobilized using a solid support to which the quantification moiety binds. For example, the solid support can include surface-bonded moieties which interact, covalently or non-covalently, with the quantification moiety. When the quantification moiety is biotin, for example, suitable surface-bonded moieties include avidin and streptavidin, which can be linked to the surface of beads, plates and other solid supports as is known in the art. The solid support is then preferably washed to remove any background signal. The immobilized complex can then be quantitated using, for example, an enzyme-linked immunosorbent assay (ELISA). In the embodiment illustrated in FIG. 1, MetAP-2-forms a complex with a biotinylated fumagillin analogue and the resulting MetAP-2/inhibitor complex is captured on a streptavidin bead. The immobilized complex is contacted with an anti-MetAP-2 antibody followed by a secondary antibody. The results can be compared to standard curve using isolated MetAP-2.

In an alternative embodiment, the MetAP2/inhibitor complex is captured with an immobilized anti-MetAP-2 antibody and then contacted with a avidin-or streptavidin-labeled detection moiety. The biotinylated fumagillin derivative will then complex the avidin or streptavidin group thereby coupling the detection moiety to the complex. For example, a fluorescent tag or radionuclide can be attached to the avidin or streptavidin. In another embodiment, the MetAP-2/inhibitor complex is separated from any unreacted quantifiable MetAP-2 inhibitor by a suitable separation method, such as dialysis or gel filtration chromatography. The fraction which includes the MetAP-2/inhibitor complex is then analyzed via a method suitable for the quantification moiety, as is known in the art. For example, if the quantification moiety is a fluorescent group, the fluorescence intensity can be determined. If the quantification moiety is a radionuclide, the radioactivity level of the fraction can be determined.

In yet another embodiment, the invention provides a method of quantifying a compound or compounds which are irreversible inhibitors of a biological target, such as MetAP-2, in a biological sample. This method comprises the steps of (1) contacting the biological sample with a saturating amount of the biological target, whereby substantially all of the compound or compounds which are irreversible inhibitors of the biological target react with the biological target, thereby forming inactivated biological target and free biological target; and (2) determining the amount of free biological target in the biological sample.

In one embodiment, the amount of free biological target is determined by measuring the activity, such as the enzyme activity or binding activity, of the biological target.

In another embodiment, the amount of free biological target is determined by a method comprising the steps of (i) contacting the biological sample with a saturating amount of a quantifiable irreversible inhibitor of the biological target, whereby substantially all of the free biological target in the biological sample reacts with the quantifiable irreversible inhibitor to form a target/inhibitor complex; (ii) determining the amount of target/inhibitor complex produced in step (i); and (iii) comparing the amount of target/inhibitor complex determined in step (ii) with the total amount of biological target added in step (1), wherein a decrease in the amount of target/inhibitor complex determined in step (ii) compared to amount of biological target added in step (1) indicates the amount of a compound or compounds in the biological sample which are irreversible inhibitors of the biological target.

In this embodiment, the biological target is present in a saturating amount if it is present in molar excess over the anticipated amount of irreversible inhibitor in the biological sample. The biological target can, for example, be present at a 1.1- to 10-fold molar excess over the anticipated amount of the irreversible inhibitor. The anticipated amount of irreversible inhibitor can be determined, for example, using chromatographic determination of the inhibitor/inhibitor complex. For the operation of the inventive method, it is not necessary that every molecule of the irreversible inhibitor react with the biological target, but the amount that reacts with the biological target should be large compared to the amount which does not, i.e., greater than about 50% of the irreversible inhibitor should react with the biological target, preferably greater than about 60%, and more preferably greater than about 75% and most preferably greater than about 90%.

The irreversible inhibitor can be a single molecular species, or a combination of two or more species. For example, the irreversible inhibitor can be the test compound administered to the subject in vivo, one or more active metabolites of the test compound or a combination thereof.

The biological sample can be a biological sample removed from a subject, for example, a subject to which a test compound can be administered in vivo, or a sample used in an in vitro assay, such as a cell-based assay or cell-free assay. For example, the biological sample can comprise liver microsomes in vitro, and the method can be used, for example, to determine the total inhibitor activity remaining after incubating a test compound with the liver microsomes. After such incubation, activity could be due to the parent compound, one or more active metabolites, or a combination thereof.

The present methods can also be combined with other analyses of the biological sample, such as flow cytometry, immunohistochemistry, gel electrophoresis/western blotting, capture of soluble molecules via ELISA. For example, extra- or intra-cellular proteins on one or multiple cell types within a biological sample can be contacted with antibodies labeled with fluorescent molecules detectable by a flow cytometer. Analysis of the data, for example, can determine changes in the numbers or types of cells within the biological sample, changes in the level of molecule expression on the surface and/or interior surface of a cell within the biological sample, the stage of replication of a cell within the biological sample. Preferred types of biological samples are derived from whole blood, bone marrow, lymph nodes, spleen, thymus, or any area of angiogenesis or inflammation. Suitable examples of molecules whose expression can be investigated include CD3, CD4, CD8, CD11a, CD11b, CD19, CD24, CD25, CD26, CD34, CD43, CD44, CD45R, CD45RA, CD45RB, CD45RO, CD62L, CD71, CD117, CD127, CXCR4, and DNA.

This invention is further illustrated by the following examples which should not be construed as limiting. The contents of all references, patents and published patent application cited throughout this application, as well as the figures are hereby incorporated by reference.

### EXAMPLES

### Example 1

### Synthesis of the Biotinylated Fumagillin Analog of Formula II (Compound 1)

1.8 mmole (1.2 equiv.) Fmoc-Lys(Biotin)-OH was dissolved in 10 mL dry DMF. 4.8 eq DIEA was added, and the solution was heated gently until the carboxylic acid went in solution. The solution was then cooled to room temperature. In an oven-dried round bottom flask, 1.5 mmole of 2-chlorotrityl chloride resin (Advanced Chem Tech) was swollen in 10 mL dry dichloromethane (“DCM”). The Fmoc-Lys(Biotin)-OH solution was added to the suspended resin and shaken under dry N₂ for 4 hours. The reaction mixture was then filtered off, and the resin was rinsed with 3×3 mL DMF, 3×3 mL DCM:MeOH:DIEA (17:6:2), 3×3 mL DCM, 2×3 mL DMF, and 3×3 mL DCM. The resin was then dried over KOH under high vacuum for 2 hours. The resin loading with FMOC-Lys(Biotin) was determined to be ˜0.63 mmole/g by dibenzofulvene absorbance.

Fmoc-Ado-OH, Fmoc-Ado-OH, and Fmoc-D-Val-OH were coupled in succession on a Rainin PS-3 Peptide Synthesizer, using 20% piperidine in DMF for FMOC deprotection (2×5 min), and 5 equivalents of FMOC-amino acid/HBTU in 0.4 M NMM in DMF for couplings (1×1 h). The N-terminal Fmoc group was removed on a PS-3 using 20% piperidine in DMF (2×5 min).

0.6 mmole of compound-resin was swollen in 9 mL dry DCM. To this suspension were added 2.4 mmole (4 equiv.) O-succinimidyl-fumagillol (Fum-OSu) and 3.6 mmole (6 equiv.) triethylamine. The mixture was stirred under dry N₂ gas for 5 hours, then the reaction mixture was filtered off, the resin was washed with DCM, and the reaction was repeated with fresh reagents.

Compound 1 was cleaved from the resin (0.6 mmole) in 12 mL 30% hexafluoroisopropanol/DCM for 30 min. The cleaved product was filtered off and combined with resin washes (3×10 mL DCM). The filtrate was concentrated under reduced pressure to yield a crude product of approximately 50% purity. The crude material was purified by preparative HPLC. The yield of 96% pure compound from crude product combined from a total of 1.5 mmole starting resin was 360 mg (22%).

### Example 2

### Determination of Free MetAP-2 in Rat White Blood Cell Lysates and tissues Following Administration of Compound 2

Compound 2, used in this example and in Example 3, is the following compound:

### (1-Carbamoyl-2-methyl-propyl)-carbamic acid-(3R, 4S, 5S, 6R )-5-methoxy-4-[(2R, 3R)-2-methyl-3-(3-methyl-but-2-enyl)-oxiranyl]-1-oxa-spiro[2.5]oct-6-yl ester

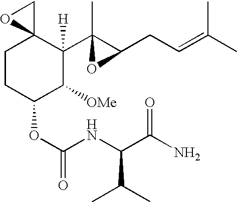

Materials

Complete Protease Inhibitor (Roche Diagnostic 1836145), 1 tablet/50 mL

EL Buffer (Qiagen 79217)

NP-40 (Calbiochem 492015)

NP-40 Lysis Buffer: 50 mM Tris pH 8.0, 150 mM NaCl, 1% NP-40

PBS: Phosphate-buffered saline, pH 7.2

RBC Lysis Buffer: Complete Protease Inhibitor resuspended in EL Buffer

WBC Lysis Buffer: NP-40 Lysis Buffer at pH 7.4 and supplemented with 0.25% sodium deoxycholate, 1 mM EDTA, 2 mM Na₃VO₄, and 1 mM NaF

Supplemented PBS wash buffer: Complete Protease Inhibitor resuspended in PBS

Polypropylene, round bottom, 96-well plates (Costar 3790)

BSA (Fraction V, Sigma A-3294)

PBST: 0.05% tween-20 in PBS

BSA/PBST: 0.2% (w/v) BSA in PBST

Reacti-Bind Streptavidin High Binding Capacity 96-well Plates (Pierce 15500)

Ethanol (AAPER 050101)

Compound 2: provided as a 40 mM solution in ethanol, stored −20° C.

Compound 1, provided as a 40 mM solution in DMSO, stored −20° C., rMetAP2

(Mediomics, 18.5 μM stock), 0.2 ng/μL in 0.2%BSA/PBST, 100 μL aliquots, stored at −20° C.

Anti-MetAP2 polyclonal antibody (Zymed 71-7200)

Goat Anti-Rabbit-HRP polyclonal antibody (Zymed 81-6120)

TMB, Peroxidase substrate (KPL 50-76-02)

TMB, Peroxidase solution B (KPL 50-65-02)

Preparation of WBC Lysates

RBC Lysis

Prepare RBC and WBC Lysis Buffers, chill on ice for at least 30 min prior to each use and use within 1 hr of the lysate preparation start time.

Transfer 0.8 mL whole blood to 15 mL conical tube on ice.

Add 4 mL ice cold RBC Lysis Buffer then invert several times and return to ice.

Incubate on ice 15 min inverting several times.

If the mixture has not become translucent after 15 min, significant RBC may still be present. Vortex briefly and incubate for another 10 min.

Centrifuge, swinging bucket rotor (1,400 RPM or approximately 400×g) 10 min at 4° C.

Decant RBC lysate supernatant and discard—gently blot tube against an absorbent pad.

Add 1.6 mL of ice cold RBC Lysis Buffer to pellet in 15 mL conical, vortex briefly.

Centrifuge as before, decant supernatant and place tube containing WBC pellet on ice

Add 3.2 mL of ice cold Supplemented PBS wash buffer, vortex briefly.

Centrifuge as before, decant supernatant and place tube containing WBC pellet on ice.

WBC Lysis

Add 0.4 mL ice cold WBC Lysis Buffer to the WBC pellet, vortex briefly.

Triturate, pipetting up & down, transfer to labeled microcentrifuge tubes and gently rock for approximately 30 min at 2-8° C.

Microcentrifuge at approximately 13.2K rpm for 10 min at 4° C.

The supernatant will be divided into 3 approximately equal aliquots into microcentrifuge tubes.

Freeze and store samples at −70° C.

Spleen Homogenization and Lysis Procedure

Prepare the Supplemented PBS, RBC Lysis Buffer and WBC Lysis Buffer, and chill on ice 30 min prior to use.

Tissue Grinding

Aliquot 2 mL Supplemented PBS into disposable tissue grinder tubes and place on ice.

Transfer freshly harvested spleens to the tissue grinder tubes and return to ice.

Homogenize the tissue then allow the samples to settle for 10 min on ice (do not centrifuge).

Cell Lysis

Decant the supernatants to 50 mL conical tubes on ice.

Add 10 mL ice-cold RBC Lysis Buffer, then invert several times and return to ice.

Incubate on ice 10 min, inverting several times.

Centrifuge in swinging bucket rotor at 400×g (1570 rpm) for 10 min at 4° C.

Decant RBC lysate supernatant and discard-gently blot tube against absorbent tissues.

Add 4 mL of ice-cold RBC Lysis Buffer to pellet in 50 mL conical, vortex briefly.

Centrifuge as before, decant supernatant and place tube containing WBC pellet on ice.

Add 10 mL of ice cold Supplemented PBS, vortex briefly.

Centrifuge as before, decant supernatant and place tube containing pellet on ice.

Add 1 mL ice-cold WBC Lysis Buffer to the WBC pellet, vortex briefly.

Triturate, transfer to silanized microcentrifuge tubes and rotate for 30 min at 4° C.

Microcentrifuge at maximum speed for 10 min at 4° C.

Aliquot the supernatants, freeze and store at −80° C.

Liver, Thymus and Lymph Nodes Homogenization and Lysis Procedure

Prepare the Supplemented PBS and chill on ice 30 min prior to use. Use dry ice to keep organ samples frozen during weighing if possible. If not, thaw samples on ice. All processing is on ice.

Weigh out 0.2 g ±0.05 g of each liver sample into disposable tissue grinders.

Add 1 mL (approximately 5 volumes) of Supplemented PBS to all and then grind the tissue until it appears homogenized.

Add 120 μL of 10% NP-40 (final concentration approximately 1%) in PBS and then allow lysis to proceed for 30 min, rotating at 4° C.

Transfer the samples to silanized microcentrifuge tubes and then microcentrifuge at maximum speed, 4° C. for 10 min.

Aliquot the supernatants and store −80° C.

ELISA

Treat 20 μL of each lysate dividing into polypropylene 96-well plates: +Compound 2 Samples (Background Controls): Dilute 40 mM Compound 2 stock 1:4000 for a 10 μM working stock and add 2 μL to background samples for a final concentration of 1 μM Compound 2.

−Compound 2 Samples receive EtOH vehicle (1 μL EtOH in 4,000 μL of PBST), add 2 μL to sample

Cover, gently tap to mix, and incubate at room temperature for 30 min.

During this time take out rMetAP2 from −20° C. and thaw. Prepare a dilution series for the standard curve: 8, 4, 2, 1, 0.5, and 0 ng/mL rMetAP2 in BSA/PBST.

Dilute 40 mM Compound 1 stock 1:4000 for a 10 μM working stock and add 2 μL of diluted to the samples and 5 μL to the rMetAP2 standards for a final concentration of 1 μM; cover, tap gently to mix, and incubate for 1 hr at room temperature without shaking.

Remove the streptavidin plates from 4° C. at least 30 min prior to use Dilute each of the samples 1:10 (180 μL into the 20 μL sample) with PBST and mix well.

Transfer 20 μL and 40 μL aliquots of the diluted samples to streptavidin plates, adding to 30 μL and 10 μL of PBST (for total volumes of 50 μL each), and mix well: duplicate aliquots of each volume for the signal samples (signal sample=−Compound 2) and single aliquots of each volume for the background samples (background samples=+Compound 2).

Transfer 20 μL aliquots of the rMetAP2 dilution series in duplicate per plate, adding each to 30 μL of PBST.

Cover and incubate the plates at room temperature on plate shaker for 1 hr at medium speed.

Wash 3 times manually with 50 μL of 2% (w/v) SDS. Flick plate briskly over sink to remove 2% SDS. Tap on napkin to blot.

Wash 4 times with 300 μL PBST, using the plate washer.

Add 50 μL of 1:500 anti-MetAP2 antibody in PBST using 12 channel pipette, so triplicates receive antibody at same time, cover and incubate at room temp. on plate shaker for 1 hr at medium speed. One plate requires 5 mL of PBST plus 10 μL of anti-MetAP2 antibody.

Wash 4 times with 300 μL PBST, using the plate washer.

Add 50 μL of 1:5000 goat anti-rabbit-HRP antibody in PBST using the 12 channel pipette, cover and incubate at room temperature on plate shaker for 1 hr at medium speed. One plate requires 5 mL of PBST plus 1 μL anti-rabbit-HRP antibody. Aliquot 50 μL per well.

Immediately following addition of HRP-conjugated antibody, turn on the plate reader and set up to read at an O.D. of 450 nm. Remove TMB solutions from 4° C. and store at room temperature until needed.

Wash 4 times with 300 μL PBST, using the plate washer, then add 50 μL of PBST to all wells in each plate and let sit for 10 minutes before starting the first plate.

One plate at a time:

Remove the PBST, invert over sink, blot on paper then add 100 μL 1:1 TMB substrate/solution B for HRP. Make enough 1:1 TMB substrate/solution B for 1 plate at a time.

10 minutes after adding the TMB substrate, add 100 μL of 1 N H₂SO₄ and then measure the absorbance at 450 nm.

### Example 3

### An Investigational Pharmacodynamic Study of Free MetAP-2 Levels in Sprague-Dawley Rats after Administration of Compound 2

Objective

The purpose of this study was to determine the percentage of free MetAP-2 remaining in white blood cells, liver, spleen, lymph nodes and thymus as a pharmacodynamic marker of Compound 2 activity after a single dose was administered to Sprague-Dawley rats.

Materials and Methods

Ninety female Sprague Dawley rats were received from Taconic Labs (Germantown, N.Y.) and used for phase I and IIa portions of this study. Sprague Dawley rats (26/sex) were received from Charles River Laboratories (Kingston, N.Y.) and used for phase IIb of the study. Animals were housed 2-3 per cage in large lexan resin cages (Allentown Caging, Allentown, Pa.) with wood chip bedding (ProChip® bedding, Harlan Inc). The commercial animal feed used was Standard Rodent Diet (#2018, Harlan Inc.) available ad libitum. A composite sample prepared from each feed lot was analyzed by the manufacturer prior to purchase. Chlorinated municipal tap water was also available ad libitum. Special analyses of feed and water were not performed since no contaminants known to be capable of interfering with the study were reasonably expected to be present. The targeted conditions for animal room temperature and humidity were 70 ±2° F., and 50 ±20%, respectively. Animals were kept on a 12 hour light/dark cycle and allowed to acclimate to the animal facility for 5 days prior to treatment.

Animals used during the study were selected on the basis of acceptable findings from pretreatment clinical observations. A random draw without replacement procedure was employed for group assignments. Each animal was identified with indelible ink on the tail and cage cards containing its unique animal number and dosage group, respectively.

Animals were monitored for survival or moribundity at least once daily during the study and body weights (to the nearest 0.1 g) were measured up to two days prior to treatment for the purpose of calculating dose volumes.

Overview of Study Design

TABLE 1
Phase I Study Design Summary

| | | | | Concen- | Dose | |
|---|---|---|---|---|---|---|
| | Test | N | Dose | tration | Volume | |
| Group | Article | Female | (mg/kg) | (mg/mL) | (mL/kg) | Route |
| 1 | Naive control* | 18** | 0 | 0 | 0 | N/A |
| 2 | Compound 2 | 18** | 30 | 5 | 6 | PO |
| 3 | Compound 2 | 18** | 30 | 5 | 6 | IV |
| 4 | Compound 2 | 18** | 30 | 5 | 6 | SC |
| 5 | Compound 2 | 18** | 30 | 5 | 6 | IP |

*To establish baseline MetAP-2 levels
**6 subgroups of 3 animals ea. (Subgroup 1-4 hr time point, Subgroup 2-24 hr time point, Subgroup 3-48 hr time point, Subgroup 4-72 hr time point, Subgroup 5-96 hr time point, Subgroup 6-120 hr time point)

Objective

MetAP-2 inhibition in white blood cells (WBC) was examined after a single dose (30 mg/kg) of Compound 2 , administered either by intravenous (IV), intraperitoneal (IP), oral gavage (PO) or subcutaneous (SC) routes, to female Sprague-Dawley (SD) rats

Test Article/Formulation

Compound 2 was prepared in a solution of 0.01% Tween 80, 0.5% trehalose, 2.0% mannitol (v/v) in 5% dextrose in water (D5W). Dose retain aliquots (1 mL in duplicate) were obtained from each study phase and stored at −70° C. for possible future analysis by HPLC.

Blood Collection

A ≧1.0 mL whole blood sample was taken from 3 animals/group/time point (4, 24, 48, 72, 96, and 120 hours post dose) for MetAP-2 analysis. Each animal was bled only once by conscious jugular venipuncture. Blood was immediately placed into EDTA tubes and stored at 4-8° C. Two blood smears from each sample were made for possible differential count analysis.

TABLE 2
Phase IIa Study Design Summary

| | | | | Concen- | Dose | |
|---|---|---|---|---|---|---|
| | Test | N | Dose | tration | Volume | |
| Group | Article | Female | (mg/kg) | (mg/mL) | (mL/kg) | Route |
| 1 | Naive | 18** | 0 | 0 | 0 | N/A |
| | control* | | | | | |
| 2 | Compound 2 | 18** | 0.3 | 0.05 | 6 | PO |
| 3 | Compound 2 | 18** | 3.0 | 0.5 | 6 | PO |
| 4 | Compound 2 | 18** | 30 | 5 | 6 | PO |
| 5 | Compound 2 | 18** | 3.0 | 0.5 | 6 | IV |

*To establish baseline MetAP-2 levels
**6 subgroups of 3 animals ea. (Subgroup 1-4 hr time point, Subgroup 2-24 hr time point, Subgroup 3-48 hr time point, Subgroup 4-72 hr time point, Subgroup 5-96 hr time point, Subgroup 6-120 hr time point)
Note:
Animal group/sub-group assignments were identical to those used in study phase I. A 10 day washout period was observed before treatment was initiated for phase IIa.

Objective

A repeat examination of MetAP-2 inhibition in WBCs was conducted with various dose levels of Compound 2, administered either IV or PO in female SD rats. In addition, thymus and liver were collected and snap frozen in liquid nitrogen for MetAP-2 analysis.

Test Article/Formulation

Compound 2 was prepared in a solution of 0.01% Tween 80, 0.5% trehalose, 2.0% mannitol (v/v) in water for injection (WfI). Dose retain aliquots (1 mL in duplicate) were obtained from each study phase and stored at −70° C. for possible future analysis by HPLC.

Blood Collection

At 4, 24, 48, 72, 96, 120 hours post dose whole blood (≧3.0 mL) was taken from anesthetized animals (isoflurane inhalation to effect) via cardiac puncture, using a 20 gauge needle. Blood was immediately placed into EDTA tubes and stored at 4-8° C.

Tissue Collection

After blood collection animals were sacrificed by CO₂ inhalation. The entire thymus and left lateral lobe of the liver were minced, placed into separate tissue cassettes, and snap frozen in liquid nitrogen for future MetAP-2 analysis.

TABLE 3
Phase IIb: Study Design Summary

| | | | | Concen- | Dose | |
|---|---|---|---|---|---|---|
| | Test | | Dose | tration | Volume | |
| Group | Article | N/Sex | (mg/kg) | (mg/mL) | (mL/kg) | Route |
| 1 | Naive | 4^{d)} | 0 | 0 | 0 | N/A |
| | control^{a} | | | | | |
| 2 | Compound 2 | 10^{b,c} | 0.3 | 0.05 | 6 | PO |
| 3 | Compound 2 | 10^{b,c} | 3.0 | 0.5 | 6 | PO |

^{a}To establish baseline MetAP-2 levels
^{b}4 subgroups of 2/sex (Subgroup 1-4 hr time point, Subgroup 2-8 hr time point, Subgroup 3-24 hr time point, Subgroup 4-48 hr time point (tissue and blood collections)
^{c}The remaining 2 animals/sex/group (subgroup 5) had blood collected at 72, 96 and 120 hours post dose
^{d)}Blood was collected from these animals as in subgroup 5 and sacrificed at the 120 hour time point

Objective

To characterize MetAP-2 turnover in tissues after a single oral administration of Compound 2 and to determine if any sex difference existed.

Results

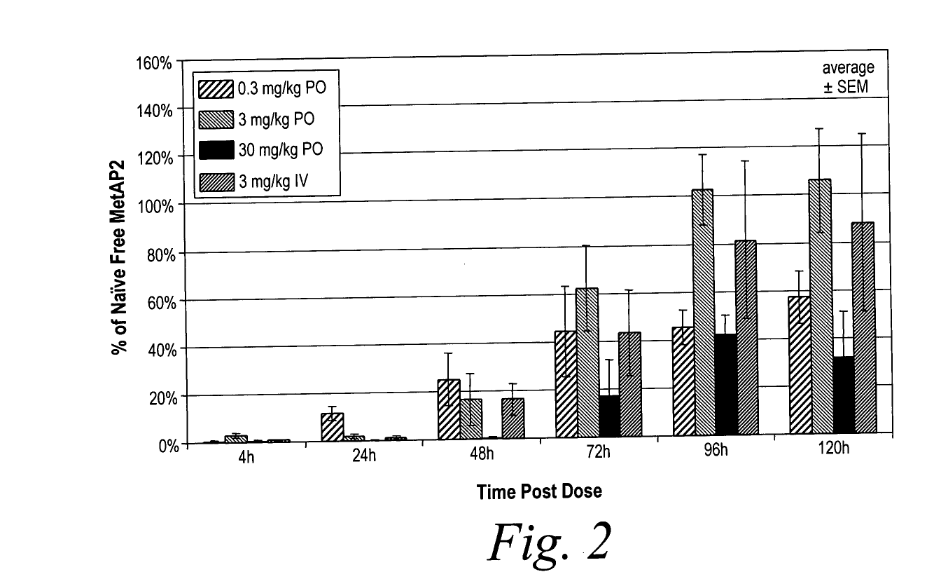

Blood and tissue samples were collected from SD rats after receiving a single dose of Compound 2. Inhibition of MetAP-2 by Compound 2 was monitored using an ELISA designed to measure the amount of free MetAP-2 in a sample. Cells were lysed and then treated with Compound 1. Compound 1 covalently binds to the active site of MetAP-2 molecules that have not already been derivatized by Compound 2. The resulting biotinylated MetAP-2 is captured onto immobilized streptavidin, then detected with an anti-MetAP-2 antibody and an enzyme-linked secondary antibody. Phase I was used as a pilot study to determine if MetAP-2 inhibition could be monitored in female SD rat white blood cell (WBC) lysates after a single 30 mg/kg dose of Compound 2 administered IV, IP, PO or SC. The ELISA was able to detect a reduction followed by a recovery of free MetAP-2 signal with all routes of administration. Following this analysis it was determined that signal from sample replicates were highly variable and the assay required revision. The ELISA format was then switched from streptavidin beads to plates and a rigorous wash with 2% sodium dodecyl sulfate (SDS) was added after the biotinylated MetAP-2 capture step. These changes reduced background signals and greatly improved the precision of the assay. Subsequent analyses for Phase IIa and IIb were conducted using the protocol set forth in Example 2. Phase IIa investigated single doses of Compound 2 at 0.3, 3 and 30 mg/kg PO or 3 mg/kg IV in female SD rats. Animals were bled and then sacrificed at 4-120 hr after dosing. Liver and thymus samples were taken for analysis methods development to be used in the next arm of the study. FIG. 2 shows the free MetAP-2 signal in WBC lysates from each dose group, given as the average free MetAP-2 in each dose group as a percentage of average naive group values. The duration of inhibition was generally related to the dose, with 30 mg/kg PO producing a more prolonged inhibition of MetAP-2 than the two lower oral doses. Administration of 3 mg/kg IV produced results that were similar to 3 mg/kg PO and had a noticeably less durable response than 30 mg/kg PO.

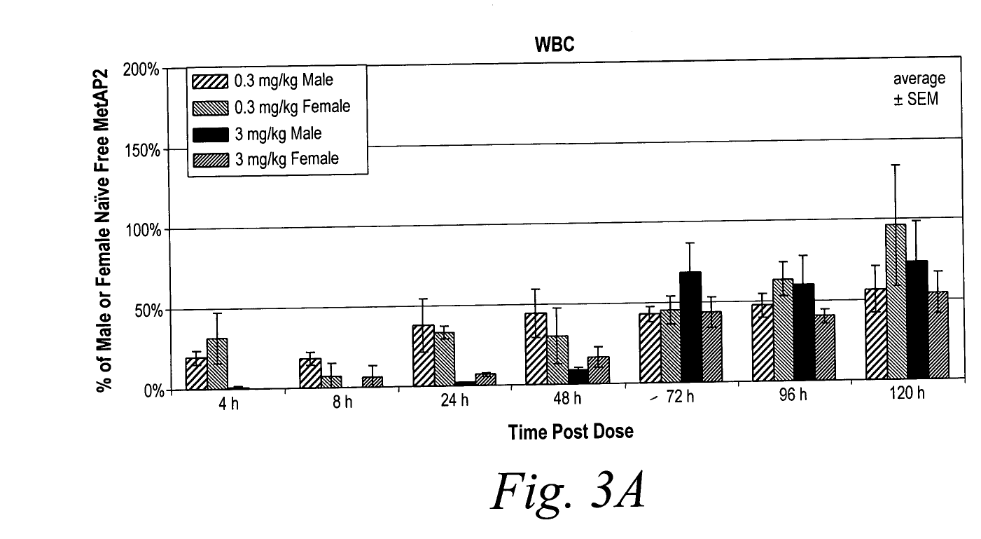

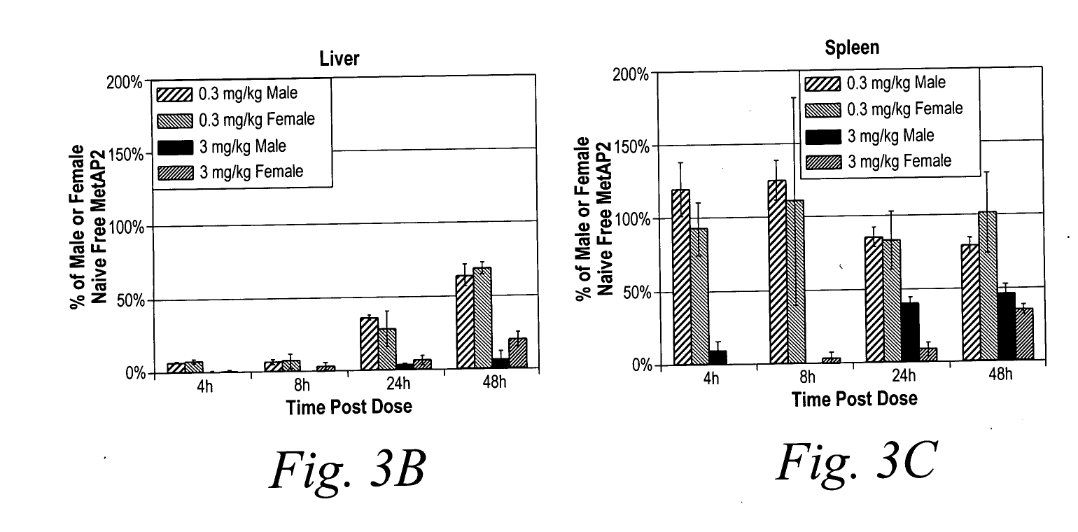

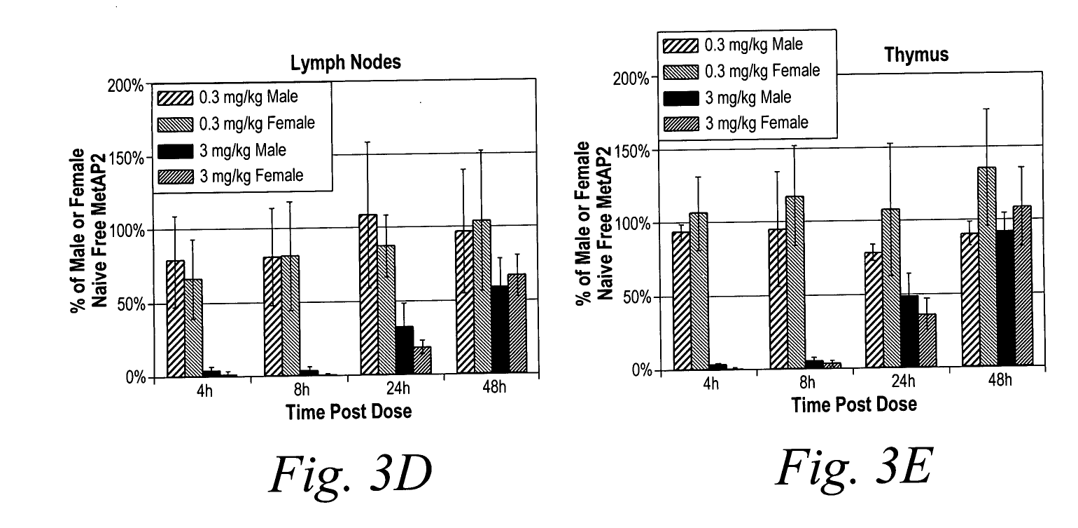

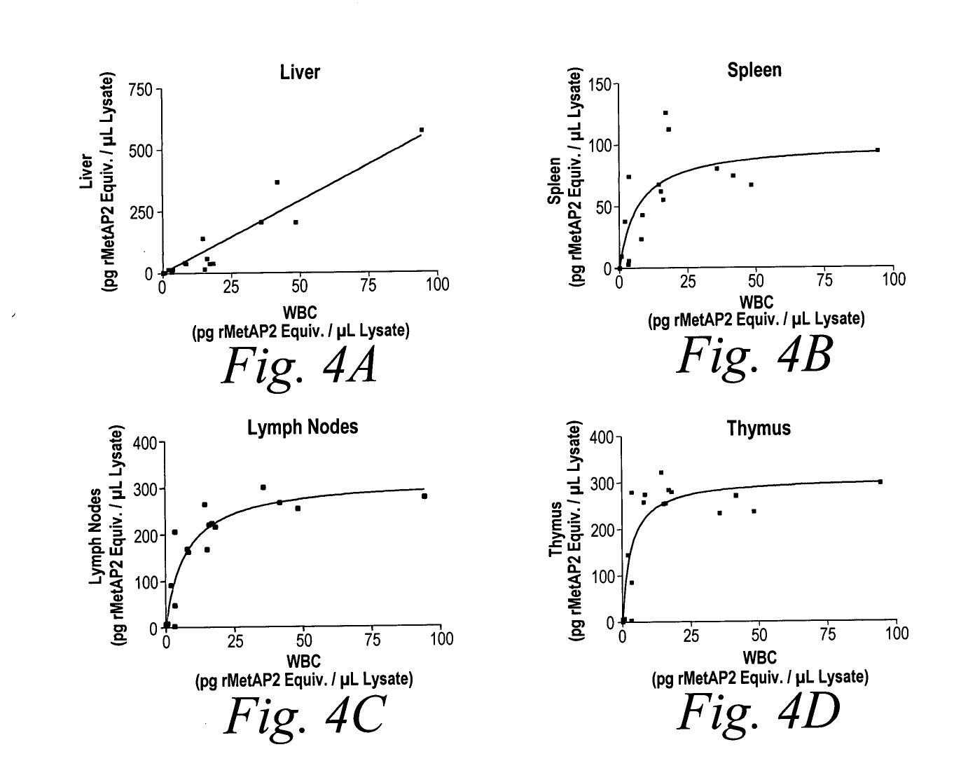

In Phase IIb, single PO doses of Compound 2 at 0.3 and 3 mg/kg were used to explore the inhibition and recovery of MetAP-2 in several organs, and compare effects in male and female SD rats. FIG. 3 shows the percentage of free MetAP-2 remaining in WBC, liver, spleen, thymus and lymph nodes at 4-48 hr after dosing. There were no consistent sex differences in MetAP-2 inhibition by Compound 2 . WBC and liver free MetAP-2 levels were distinctly more reduced than in the other tissues, where 0.3 mg/kg had no significant effect. This could reflect differences in tissue sensitivity or the level of exposure to Compound 2 in each compartment. As in Phase IIa, the inhibition in WBC from the 3 mg/kg group were initially lower than those that received 0.3 mg/kg, but the two groups had recovered to similar free MetAP-2 levels by 72 hr. Four hours after receiving 3 mg/kg Compound 2, there was an average of 95% or greater inhibition of MetAP-2 in all compartments. At 48 hr after receiving 3 mg/kg, free MetAP-2 levels in thymus tissue had recovered completely, and lymph nodes, spleen, liver and WBC were at average values (±SEM) of 63%±16%, 41%±5%, 13%±5%, 13%±4% respectively. In FIG. 4, free MetAP-2 signal in the tissues was plotted relative to those in WBC to examine the correlations between these compartments. The curves shown were fit to the data using nonlinear regression analysis. The extent of MetAP-2 inhibition in WBC required to observe inhibition in the organs was an indication of the responsiveness of each to Compound 2: liver (most inhibited)>spleen≅lymph nodes>thymus. In all cases, when a group had no measurable free MetAP-2 in the WBC, the tissues had an average of 3% or less remaining.

Conclusions

The pharmacodynamics of the inhibition of MetAP-2 by Compound 2 have been measured using an ELISA to determine the amount of free MetAP-2 present in blood and tissue samples after single doses of Compound 2 were administered to SD rats. The duration of MetAP-2 inhibition in WBCs and organs was related to the dose of Compound 2 administered by PO, and 3 mg/kg IV produced results that were similar to 3 mg/kg PO. There were no consistent sex differences in MetAP-2 inhibition by Compound 2 . Inhibition in the organs ranked (in order of decreasing response): liver>spleen≅lymph nodes>thymus. Compound 2 doses that left no measurable free MetAP-2 in WBC resulted in 3% or less remaining in tissues.

Abbreviations:

| | |
|---|---|
| BSA | Bovine Serum Albumin |
| DMSO | Dimethylsulfoxide |
| ELISA | Enzyme-Linked Immunosorbent Assay |
| Equiv. | Equivalents |
| EtOH | Ethanol |
| N | Number |
| PBMCs | Peripheral Blood Mononuclear Cells |
| PI | Protease Inhibitor |
| QOD | Every Other Day |
| rMetAP-2 | Recombinant Methionine Aminopeptidase Type-2, Human |
| SD | Standard Deviation |
| SDS | Sodium Dodecyl Sulfate |
| SEM | Standard Error of Measurement |
| TMB | 3,3′,5,5′-Tetramethylbenzidine |

### Example 4

### Analysis of Free MetAP-2 in Biological Samples Using a Fluorescence-labeled Fumagillin Analogue

Preparation of Fluorescent-labeled Fumagillin Analogue

The fluorescent labeled fumagillin analogue shown below (“Compound 3”) was prepared using solid phase synthesis as in Example 1, with a final addition of Cy5 N-hydroxysuccinimidyl ester (Amersham Biosciences) to the lysine E-nitrogen atom.

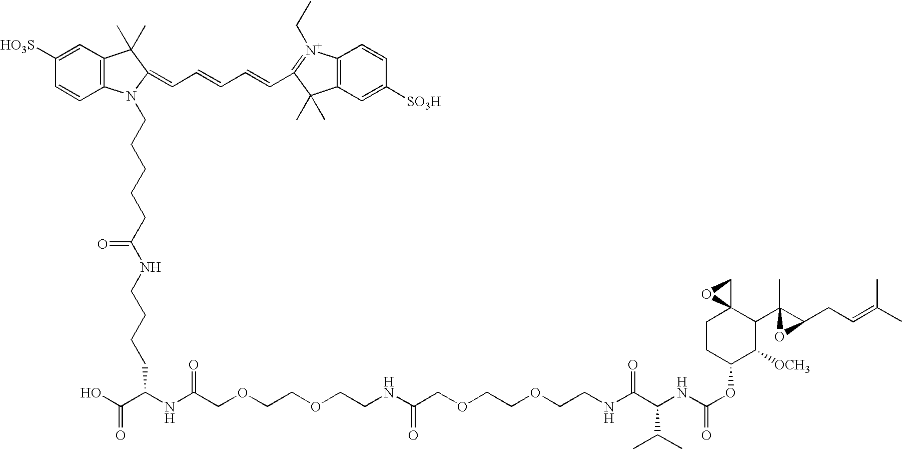

Tumor Implantation and Dosing of Mice

Male C57BL/6 mice were divided into six groups of 10 mice each. Each mouse received an implant of 10⁶B16F10 murine melanoma cells in 100 μL of PBS above the leg. At day seven following implantation, one group of mice (Group 6) began a regimen of 100 mg/kg Compound 2 every other day, administered oral gavage (PO). At day 13 post implantation, the remaining groups began receiving treatment as follows: Group 1: 5 mL vehicle (11 % hydroxypropyl cyclodextrin) every other day; Group 2: 5-fluorouracil 50 mg/kg in 1% propylene glycol/D5W, PO every other day; Group 3: Compound 2, 3 mg/kg PO every other day; Group 4: Compound 2, 30 mg/kg PO, every other day; Group 5: Compound 2, 100 mg/kg PO, every other day. In each group, the last dose was administered on day 19 post implantation, and blood, spleen, tumor, thymus and liver samples were collected from the mice 24 hours following the last dose.

Analysis of Samples

The tissue samples were prepared for analysis following the protocols set forth in Example 2 and analyzed for free MetAP-2 using the ELISA protocol of Example 2. The prepared tissue samples were also analyzed for free MetAP-2 activity using the following protocol.

Materials:

10% NuPAGE BIS-TRIS gel, 1.0 mm×15 well, catalog number NP0303, lot number 2081931, expiration date: 18 Dec. 2003

20×NuPAGE MOPS running buffer, catalog number NP001-02, lot number 222245, expiration date: 05 May 2002, diluted to 1×with milliQ water

Storm/ImageQuant for Cy5 reading, Red 635 nm/650LP

Storm/ImageQuant for Sypro Orange reading, Blue 450/520LP

Anti-Oxidant, NuPAGE, catalog number NP0005

rMetAP2, Mediomics, 18.53 uM stock=1 mg/mL

Compound 3, 0.8 mg (entire tube), FW=1483.2, 64% pure, resuspended in 345 uL of ethanol for a final concentration of 1 mM (purity adjusted)

Sypro Orange, Molecular Probes, catalog number S-6651

Lysis buffer (150 mM NaCl, 50 mM Tris-HCl, pH 8.0, 1% NP-40)

See Blue MW markers, Invitrogen, catalog number LC5925

Procedure:

1. Dilute samples in 1.5 mL eppendorf tubes to their desired concentration in lysis buffer in 10 uL volume. See calculations.

2. To each tube, add 1 uL of 10 uM Compound 3stock that has been diluted in lysis buffer.

3. Incubate on ice for 1.0 hours.

4. Turn on heat block to 70° C.

5. Add 3.8 uL of 4×sample buffer to each tube.

6. Add 1.5 uL of DTT (Novex solution)

7. Boil the tubes for 5 minutes at 70° C. Briefly spin down tubes.

8. Peel off bottom seal from two gels.

9. Outline the wells of the gels with a VWR lab marker and then remove the comb.

10. Prepare 800 mL of running buffer.

11. Place gel into apparatus, and add 0.5 mL of anti-oxidant to the center chamber. Fill the chamber with 1×running buffer.

12. Flush out all wells of the gel with running buffer before loading the samples into the wells.

13. Run the gels for 60 minutes at 200V, room temperature

14. After running the gel, stain the blot with Sypro Orange as follows:

15. Wash the gel for 10 minutes with milliQ water.

16. Add Sypro Orange (see calculations for dilution)

17. Cover the gel box with foil, and incubate shaking for one hour.

18. Quickly rinse the gel with 7.5% acetic acid.

19. Wash the gel with 7.5% acetic acid.

20. Scan the gel on the Storm with both the 450 nm filter (Sypro Orange) and at 635 nm (Cy5) simultaneously.

21. Analysis in ImageQuant: Under view, choose Multichannel, select side by side gray scale to see scans of each individual wavelength.

| | |
|---|---|
| rMetAP2 | stock is 18.53 uM = 18,530 nM |
| | 1:100 = 185.3 nM |
| | 1:1,000 = 18.53 nM |

| | | |
|---|---|---|
| conc. in 10 uL | volume of dilution | uL volume of lysis buffer |

| | | |
|---|---|---|
| 10 nM | 0.5 uL of 1:100 | 9.5 |
| 1 nM | 0.5 uL of 1:1,000 | 9.5 |

Lysates: Use the ELISA guidelines for volume of sample to load.

| buffer | ELISA guideline | uL per well | uL lysis |
|---|---|---|---|
| Wbc: | 2-4 uL per well | 4 | 6 |
| Liver: | 0.2-1 uL per well | 1 | 9 |
| Spleen: | 1-2 uL per well | 2 | 8 |
| Thymus: | 2-4 uL per well | 4 | 6 |
| Tumor: | 0.2-1 uL per well | 1 | 9 |

Sypro Orange: dilute the stock Sypro reagent 1:5,000 in 7.5% (v/v) acetic acid (2 uL in 100 mL)

Compound 3: 1 mM stock=1000 uM; 1:100=10 uM. Dilute in the sample reactions (1 uL 10 uM+10 uL, not quite 1:10 but 1:11) to yield a 1 uM stock.

For 100 nM: dilute the stock 1:1000=1 uM or 1000 nM, Add 1 uL to the rMetAP2 reaction.

Results

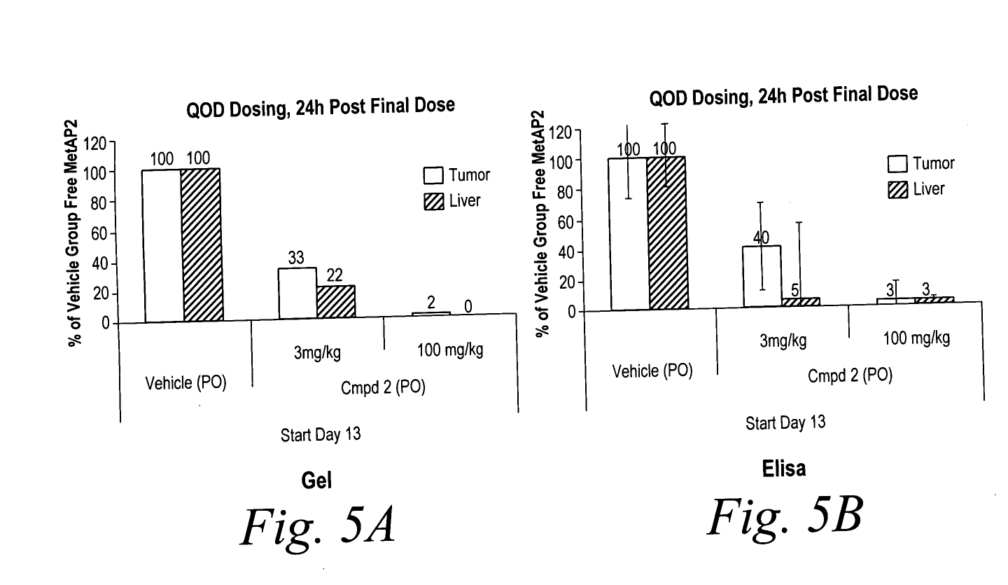

The results of this study are set forth in FIG. 5, which provides a comparison of the results obtained in tumor tissue and liver tissue using the ELISA protocol and those obtained using the gel-shift analysis. In all cases a dose-dependent decrease in free MetAP-2 levels is seen in both tissues relative to the controls.

### Example 5

### Determination of Free MetAP-2

This Example describes a free MetAP-2 ELISA protocol which is an alternate to the protocol set forth in Example 2.

Materials:

Biotin (Pierce 29129), 2.34 mM stock in DMSO, 100 μL aliquots stored −20 C, was prepared fresh each month.

Compound 1, 1.17 mM stock in DMSO, 50 μL aliquots stored −20 C, was prepared fresh every 3 months.

Compound 1-rMetAP2, 234 μg/mL in 20 mM HEPES pH 7.3, 150 mM NaCl, 10% Glycerol, 0.1 mM CoSO₄, (KFW-1035-001), −20° C.

Reacti-Bind Streptavidin High Binding Capacity 96-well Plates (Pierce 15500).

Polypropylene, round bottom, 96-well plates (Costar 3790).

1.7 mL Polypropylene microcentrifuge tubes (VWR 20170-038 or equivalent).

15 mL Conical polypropylene centrifuge tubes (VWR 21008-103 or equivalent).

50 mL Conical polypropylene centrifuge tubes (VWR 20171-038 or equivalent).

PBST (PBS+0.05% Tween-20)

2% (w/v) SDS (Sodium Dodecyl Sulfate)

Anti-MetAP-2 polyclonal antibody (Zymed 71-7200)

Goat Anti-Rabbit-Horse radish peroxidase polyclonal antibody (Zymed 81-6120).

TMB, Peroxidase substrate (KPL 50-76-02)

TMB, Peroxidase solution B (KPL 50-65-02)

Plate shaker (Lab-Line Instruments, Inc., Model 4625)

Plate washer (BIO-TEK Instruments Inc., ESx 405 Select)

Solution Preparation:

1. Matrix: 25 mL of 20%, 1% or 0.02% naïve lysates (depending on sample types and dilutions to be run) was prepared by diluting into PBST.

2. Biotin:

a. 2.19 μM Biotin solution was prepared by adding 37.5 μL of 2.34 mM Biotin stock to 40 mL of PBST in a 50 mL conical tube.

b. 438 nM Biotin was prepared in Matrix solution by adding 6 mL of 2.19 μM Biotin solution to 24 mL of 20%, 1% or 0.02% of Matrix in a 50 mL conical tube.

3. Compound 1: 438 nM solution of Compound 1 was prepared by adding 15 μL of 1.17 mM Compound 1 stock to 40 mL of PBST in a 50 mL conical tube.

4. Standard Solutions: One aliquot of 10 μg/mL Compound 1-rMetAP-2 was thawed as a standard working stock.

a. If a new 10 μg/mL standard working stock was needed, it was prepared in a polypropylene microcentrifuge tube by thawing an aliquot of 234 μg/mL Compound 1-rMetAP-2 and adding 20 μL of it to 448 μL of 438 nM Biotin, pipetting up and down then inverting several times to mix well without foaming. The solution was divided into 15 μL aliquots and frozen at −70 C.

b. The working stock was diluted to 500 ng/mL by adding 10 μL of it to 190 μL of 438 nM Biotin, pipetting up and down then inverting several times to mix well without foaming.

c. Standard solutions 1-10 were prepared by further serial dilution into 438 nM Biotin in Matrix, each time pipetting up and down then inverting several times to mix:

| | | 438 nM | | | |
|---|---|---|---|---|---|
| Standard | | Biotin | | | |
| Solution | | in | Compound 1-rMetAP2 | | |
| Concentration | Calibrator | Matrix | Vol. | | Std |
| (ng/mL) | Type | (μL) | (μL) | Solution | ID |
| 20.0 | High Anchor | 480 | 20 | 500 ng/mL | S-1 |
| 10.0 | Quantitation | 200 | 200 | S-1 (20 ng/mL) | S-2 |
| 5.00 | Quantitation | 200 | 200 | S-2 (10 ng/mL) | S-3 |
| 2.00 | Quantitation | 240 | 160 | S-3 (5 ng/mL) | S-4 |
| 1.00 | Quantitation | 200 | 200 | S-4 (2 ng/mL) | S-5 |
| 0.500 | Quantitation | 200 | 200 | S-5 (1 ng/mL) | S-6 |
| 0.200 | Quantitation | 240 | 160 | S-6 (0.5 ng/mL) | S-7 |
| 0.100 | Quantitation | 200 | 200 | S-7 (0.2 ng/mL) | S-8 |
| 0.0500 | Low Anchor | 200 | 200 | S-8 (0.1 ng/mL) | S-9 |
| 0.0200 | Low Anchor | 240 | 160 | S-9 (0.05 ng/mL) | S-10 |

ELISA:

1. Preparation of the lysate test samples at final dilutions of 1:5, 1:10, 1:50, 1:100, 1:500, 1:1000 and 1:5000.

a. Test samples were removed from frozen storage and allow to thaw at room temperature.

b. Intermediate dilutions were prepared for the samples in PBST as follows in a polypropylene 96-well plate or eppendorf tubes for the two part dilutions:

1:5-40 μL PBST+40 μL sample

1:10-60 μL PBST+20 μL sample

1:50-76 μL PBST+4 μL sample

1:100-78 μL PBST+2 μL sample

1:500-1:10 (90 μL PBST+10 μL sample); 1:50 (76 μL PBST+4 μL 1:10 sample)

1:1000-1:10 (90 μL PBST+10 μL sample); 1:100 (76 μL PBST+2 μL 1:10 sample)

1:5000-1:100 (990 μL PBST+10 μL sample); 1:50 (76 μL PBST+4 μL 1:10 sample)

Mix well by pipetting up and down several times.

c. 20 μL of 2.19 μM biotin was added to all samples and mixed well by pipetting up and down several times.

d. 100 μL of 438 nM Compound 1 was added to all samples and mixed well by pipetting up and down several times.

2. 200 μL of each standard was transferred to empty wells in the polypropylene plate.

3. The plates were covered and incubated at room temperature for 1 hr.

Streptavidin plates were removed from 4° C. at least 30 min prior to use.

4. Capture on streptavidin plates:

a. Streptavidin plates were washed 4 times with 300 μL PBST, using the plate washer, then tappe on paper towels to remove any remaining solution.

b. 80 μL aliquots of the test samples were pippetted up and down twice, and then transferred in duplicate and standards from the polypropylene plates to the streptavidin plates.

c. Plates were covered and incubated at room temperature for 1 hr (no shaking).

5. 1:500 dilution of anti-MetAP-2 antibody in PBST was prepared.

6. Washes:

a. Plates were washed 4 times with 300 μL PBST, using the plate washer.

b. 100 μL of 2% (w/v) SDS was added. After 2 min, the solution was then aspirated using the plate washer.

c. The plate washer washer was reset using an empty plate.

d. 100 μL of 2% (w/v) SDS was added. After 2 min, the plates were washed 4 times with 300 μL PBST, using the plate washer, then tapped on paper towels to remove any remaining solution.

7. 80 μL (reset pipette) of 1:500 anti-MetAP2 antibody in PBST was added, then covered and incubated at room temperature for 1 hr.

8. 1:5000 dilution of goat anti-rabbit-HRP antibody in PBST was prepared. (Need a minimum of 8 mL per plate.)

9. Plates were washed 4 times with 300 μL PBST, using the plate washer, then tapped on paper towels to remove any remaining solution.

10. 80 μL of 1:5000 goat anti-rabbit-HRP antibody in PBST was added. Plate was covered and incubated at room temperature for 1 hr.

Immediately following addition of HRP-conjugated antibody, the plate reader was turned on and set up to read at an O.D. of 450 nm. TMB solutions were removed from 4° C. and stored at room temperature until needed.

11. Quantitate with TMB substrate:

a. 1:1 TMB substrate/solution B was prepared. Plates were washed 4 times with 300 μL PBST (using the plate washer), then tapped on paper towels to remove any remaining solution.

b. 100 μL 1:1 TMB substrate/solution B was added for HRP.

c. 10 minutes after adding the TMB substrate, 100 μL of 1 N H₂SO₄ was added and then the absorbance at 450 nm was measured.

Equivalents

Those skilled in the art will recognize, or be able to ascertain using no more that routine experimentation, many equivalents to the specific embodiments of the invention described herein. Such equivalents are intended to be encompassed by the following claims.

We claim:

1. A method of measuring the ability of a test compound to inactivate a biological target in a cell of a subject, comprising the steps of:
(a) administering the test compound to a subject, such that any of the biological target in the subject's body which reacts with the test compound is inactivated and any of the biological target which does not react with the test compound is free;
(b) removing a biological sample comprising one or more cell types from the subject;
(c) determining the amount of free biological target within the biological sample or a fraction thereof; and
(d) comparing the amount determined in step (c) with the amount of free biological target in a control sample,
wherein a decrease in the amount of free biological target determined in step (c) compared to the amount determined in the control sample provides a measure of the amount of inactivated biological target in the biological sample or fraction thereof.

2. The method of claim 1 wherein the amount of free biological target is determined by measuring the activity of the biomolecule within the biological sample or fraction thereof.

3. The method of claim 1 wherein the amount of free biological target is determined by a method comprising the steps of:
(i) contacting the biological sample or a fraction thereof with a saturating amount of a quantifiable irreversible inhibitor of the biological target, so that substantially all of the free biological target reacts with the quantifiable irreversible biological target inhibitor to form a target/inhibitor complex; and
(ii) determining the amount of target/inhibitor complex formed in step (i).

4. The method of claim 1 wherein the biological target is an enzyme, a g-protein coupled receptor, a cytokine, or a receptor kinase.

5. The method of claim 4 wherein the biological target is MetAP-2.

6. A method for determining the extent of inactivation of MetAP-2 in a biological sample or fraction thereof derived from a subject, comprising the steps of:
(a) administering a test compound to the subject, wherein any MetAP-2 in the body of the subject that reacts with the test compound is inactivated MetAP-2 and any MetAP-2 that does not react with the test compound is free MetAP-2;
(b) removing a biological sample from the subject, wherein said biological sample comprises one or more types of cells; and
(c) determining the amount of free MetAP-2 in the biological sample or a fraction thereof; and
(d) comparing the amount determined in step (c) with the amount determined in a control sample;
wherein a decrease in the amount determined in step (c) compared to the amount determined in step (d) is a measure of the extent of inactivation of MetAP-2 in the biological sample or fraction thereof.

7. The method of claim 6 wherein the amount of free MetAP-2 is determined using a method comprising the steps of:
(i) contacting at least a portion of the biological sample with a saturating amount of a quantifiable irreversible MetAP-2 inhibitor, whereby substantially all of the free MetAP-2 in the biological sample reacts with the quantifiable irreversible Metap-2 inhibitor to form a MetAP-2/inhibitor complex; and
(ii) determining the amount of MetAP-2/inhibitor complex produced in step (i).

8. The method of claim 1 wherein the biological sample is selected from the group consisting of whole blood, a blood fraction, erythrocytes, white blood cells, T-cells, B-cells, macrophages; tumor tissue; cancer cells; bone marrow; synovium, synovial fluid, cerebrospinal fluid; liver tissue; brain tissue; prostate tissue, breast tissue, lymph node tissue and spleen.

9. The method of claim 1 further including the step of lysing the cells following step (b).

10. The method of claim 1 further comprising the step of homogenizing the biological sample or a portion of the biological sample following step (b).

11. The method of claim 6 wherein the test compound inhibits MetAP-2 activity in vitro.

12. The method of claim 11 wherein the test compound is an irreversible inhibitor of MetAP-2.

13. The method of claim 12 wherein the test compound is a covalent inhibitor of MetAP-2.

14. The method of claim 13 wherein the test compound is a fumagillin analogue.

15. The method of claim 1 wherein the quantifiable irreversible MetAP-2 inhibitor is a fumagillin analogue.

16. The method of claim 15 wherein the fumagillin analogue comprises a biotin moiety.

17. The method of claim 16 wherein the fumagillin analogue is of the structure:

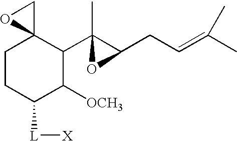

18. The method of claim 17, wherein the fumagillin analogue is of the structure:

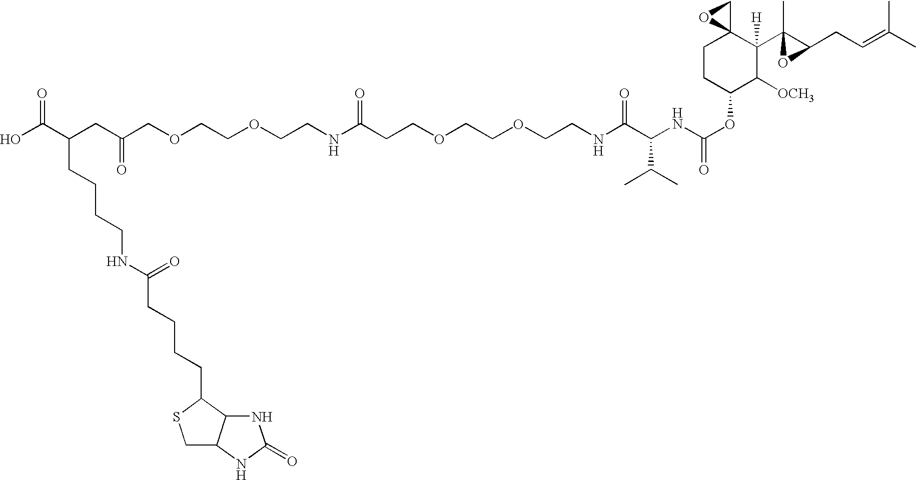

19. A method of quantifying a compound or compounds which are irreversible inhibitors of a biological target in a biological sample, said method comprising the steps of
(a) contacting the biological sample with a saturating amount of the biological target, whereby substantially all of the compound or compounds which are irreversible inhibitors of the biological target react with the biological target, thereby forming inactivated biological target and free biological target; and
(2) determining the amount of free biological target in the biological sample.

20. The method of claim 19 wherein the amount of free biological target is determined by measuring the activity of the biological target.

21. The method of claim 20 wherein the activity is enzymatic activity or binding activity.

22. The method of claim 19 wherein the amount of free biological target is determined by a method comprising the steps of:
(i) contacting the biological sample with a saturating amount of a quantifiable inhibitor of the biological target, whereby substantially all of the free biological target in the biological sample reacts with the quantifiable irreversible inhibitor to form a target/inhibitor complex;
(ii) determining the amount of target/inhibitor complex produced in step (i); and
(iii) comparing the amount of target/inhibitor complex determined in step (i) with the total amount of biological target added in step (1),
wherein a decrease in the amount of target/inhibitor complex determined in step (ii) compared to amount of biological target added in step (1) indicates the amount of a compound or compounds in the biological sample which are irreversible inhibitors of the biological target.

23. The method of claim 19 wherein the biological target is MetAP-2.

24. The method of claim 23 wherein the compound or compounds which are irreversible inhibitors of MetAP-2 are fumagillin analogues.

25. The method of claim 22 wherein the biological target is MetAP-2 and the quantifiable inhibitor is a fumagillin analogue comprising a quantification moiety.
